(19) United States
(12) Patent Application Publication
(10) Pub. No.: US 2009/0177086 A1
(43) Pub. Date: Jul. 9, 2009

(54) METHOD AND APPARATUS FOR SELECTIVELY ENHANCING ULTRASOUND IMAGE DATA

(76) Inventor: Erik Normann Steen, Moss (NO)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 11/971,688

(22) Filed: Jan. 9, 2008

(51) Int. Cl. A61B 8/06

(52) U.S. Cl. 600/443

(57) ABSTRACT

Noise may be reduced in ultrasound images that comprise at least a fluid area and a tissue area. The image data comprises pixels having input intensity values. Edge pixels associated with an edge within the tissue area are detected. The input intensity values of at least a portion of non-edge pixels are modulated to be less than the input intensity value of the none-edge pixel to form a selectively enhanced image for display.

### BACKGROUND OF THE INVENTION

This invention relates generally to ultrasound imaging, and more particularly, to reducing noise and selectively enhancing ultrasound images.

Noise reduction algorithms typically average noise, such as speckle and thermal noise, in an attempt to make the noise less apparent within the ultrasound image. In some areas of the image, such as areas having blood or other fluid, the noise is still visible as a gray “cloud”. Additionally, noise in fluid makes it difficult for the operator to see borders or edges between the fluid and the tissue.

Thresholding has also been used to reduce noise. A threshold level is typically applied to the entire image to remove or reduce low gray levels that are below the threshold level. Although the noise is removed or reduced within the fluid, thresholding also removes noise from the surrounding tissues and thus may also remove data that may be used for diagnosis.

Therefore, improving the reduction of noise within image data is desirable.

### BRIEF DESCRIPTION OF THE INVENTION

In one embodiment, a method for reducing noise in ultrasound images comprises accessing ultrasound image data comprising at least a fluid area and a tissue area. The image data comprises pixels having input intensity values. Edge pixels associated with an edge within the tissue area are detected. The input intensity values of at least a portion of non-edge pixels are modulated to be less than the input intensity value of the non-edge pixel to form a selectively enhanced image for display.

In another embodiment, a computer readable medium for selectively enhancing image data comprises instructions to access image data comprising at least a fluid area and a tissue area. The computer readable medium further comprises instructions to detect at least one edge comprising edge pixels within the tissue area and instructions to modulate intensity values associated with at least a portion of non-edge pixels to increase a contrast level between the fluid area and the tissue area.

In yet another embodiment, a method for processing image data comprises accessing image data comprising pixels having input intensity values. The input intensity values have a range based on minimum and maximum intensity values. Edge pixels associated with an edge within the image data are detected. A weight value is computed for each of the pixels based on the associated input intensity value. The weight values of at least a portion of non-edge pixels are decreased and an image is displayed wherein the pixels have output intensity values based on the weight values and the input intensity values.

### BRIEF DESCRIPTION OF THE DRAWINGS

FIG. 1 illustrates an ultrasound system formed in accordance with an embodiment of the present invention.

FIG. 2 illustrates a method for selectively enhancing ultrasound images in accordance with an embodiment of the present invention.

FIG. 3 illustrates an exemplary table of input intensity values and weight values for modulating pixels in accordance with an embodiment of the present invention.

FIG. 4 is a drawing that represents an example of cardiac scanning in accordance with an embodiment of the present invention.

FIG. 5 is a drawing that illustrates intensity weighting only of pixel intensity values in accordance with an embodiment of the present invention.

FIG. 6 is a drawing that illustrates intensity weighting, edge detection weighting and temporal filtering of pixel intensity values in accordance with an embodiment of the present invention.

FIG. 7 illustrates a 3D-capable miniaturized ultrasound system formed in accordance with an embodiment of the present invention.

FIG. 8 illustrates a hand carried or pocket-sized ultrasound imaging system formed in accordance with an embodiment of the present invention.

FIG. 9 illustrates a console-based ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

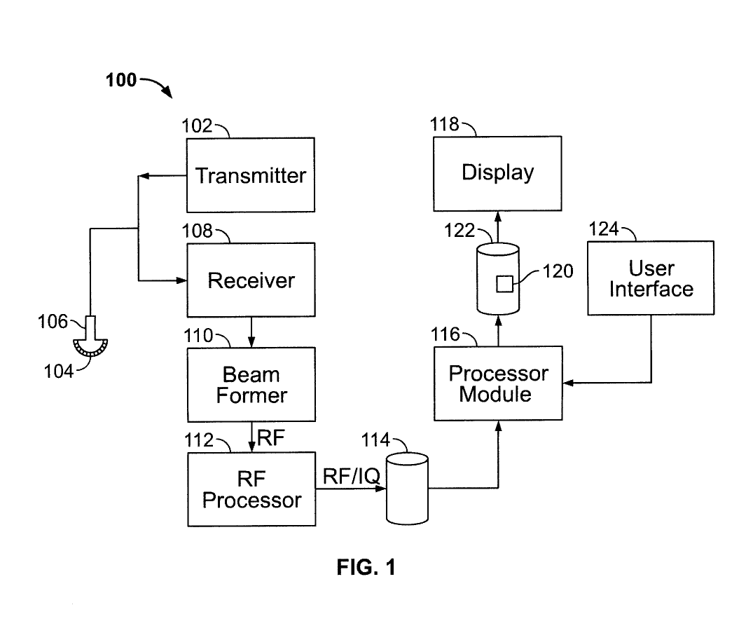

FIG. 1 illustrates an ultrasound system 100 including a transmitter 102 that drives an array of elements 104 (e.g., piezoelectric elements) within a transducer 106 to emit pulsed ultrasonic signals into a body. The elements 104 may be arranged, for example, in one or two dimensions. A variety of geometries may be used. The ultrasonic signals are back-scattered from structures in the body, like fatty tissue or muscular tissue, to produce echoes that return to the elements 104. The echoes are received by a receiver 108. The received echoes are passed through a beamformer 110 that performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 or memory 122 during a scanning session and then processed and displayed in an off-line operation.

A user interface 124 may be used to input data to the system 100, adjust settings and control operation of the processor module 116. The display 118 includes one or more monitors that present patient information, including diagnostic ultrasound images to the user for diagnosis and analysis. One or both of memory 114 and memory 122 may store two-dimensional (2D) and/or three-dimensional (3D) datasets of the ultrasound data, where such datasets are accessed to present 2D and/or 3D images. Multiple consecutive 3D datasets may also be acquired and stored over time, such as to provide real-time 3D or four-dimensional (4D) display. The images may be modified and the display settings of the display 118 also manually adjusted using the user interface 124. A selective enhancement module 120 also may be provided, for example, as part of the memory 122 and as described in more detail below. It should be noted that the selective enhancement module 120 may be provided in or as part of different portions of the ultrasound system 100, for example, as part of the processor module 116 and may be implemented in software, hardware or a combination thereof.

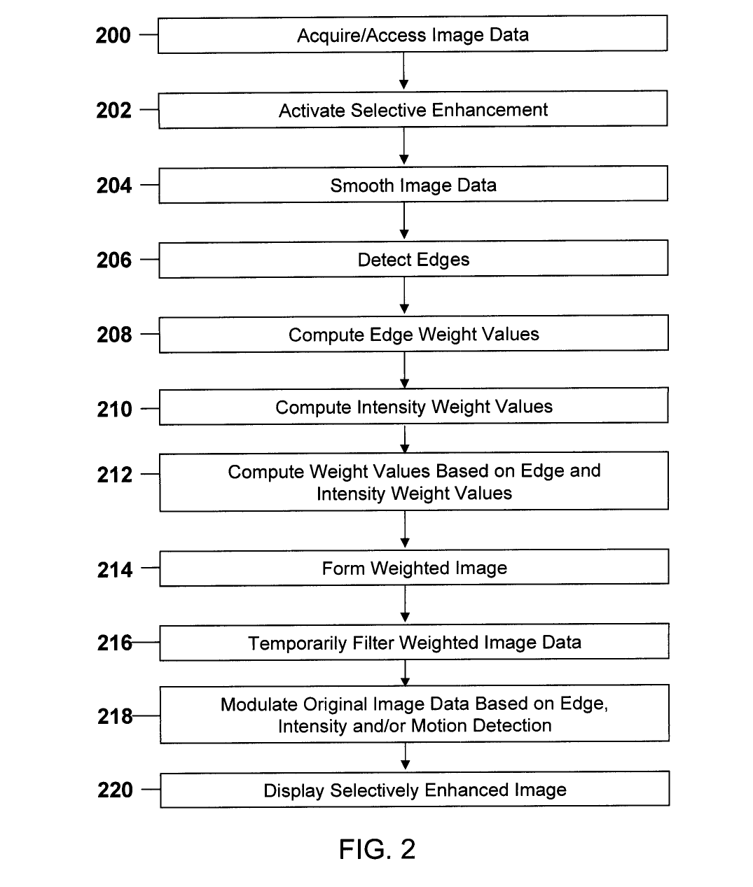

FIG. 2 illustrates a method for selectively enhancing ultrasound images, such as by adaptively reducing noise within portions of the ultrasound image. As discussed previously, segmentation has previously been used to reduce the noise level over the entire image. While reducing the noise in areas having fluid, such as within the ventricles of the heart, segmentation also reduces intensity of image data associated with tissue and thus important image data may be lost. By adaptively or selectively reducing noise within the image, noise may be suppressed in areas having fluid while areas associated with tissue may be unchanged or enhanced. This may improve a contrast level between the fluid and tissue, improving the visualization of the tissue edges.

At 200, ultrasound image data may be accessed and/or acquired. For example, the system 100 of FIG. 1 may be used to acquire ultrasound data of a patient's heart. The image data may be processed and displayed in real-time while the patient is being scanned or after the image data has been acquired and stored, such as in the memory 122. The image data may be 2D or 3D, a single frame of image data or multiple frames acquired over time, such as 2D or 3D image data over time. 3D image data over time may also be referred to as 4D image date. The image data may comprise a plurality of pixels, each of which has an associated input intensity value.

At 202 the processor module 116 activates the selective enhancement module 120. For example, the operator may use the user interface 124 to select a key, input a voice command, select a graphical user input (GUI) location on a touchscreen and the like, to activate the selective enhancement module 120. In another embodiment, the selective enhancement module 120 may be automatically activated from within a protocol selected by the operator.

At 204 the processor module 116 may smooth the image data of 200 to generate a smoothed image. Smoothing may be accomplished across the entire image, such as by acting upon each pixel or group of pixels within the image data. The processor module 116 may smooth the image, for example, by averaging neighboring pixels, applying a speckle reduction algorithm, and/or by using a different smoothing operation. The smoothing reduces the local variance of at least a portion of the pixels within the image data. The smoothing may be applied to one or more frames of image data, such as the frames of image data over time, if more than one image frame is being processed. The smoothed image may be stored in the memory 122 and is not displayed on the display 118. In one embodiment, smoothing may decrease the variation of the image data so that fewer false edges may be detected. In another embodiment, the smoothing of 204 may be optional.

At 206 the processor module 116 detects edges (or edge pixels) within one of the smoothed image data and the original image data of 200. In other words, the processor module 116 may compute, for every pixel within the smoothed image data, whether an edge is present. For example, an edge may be an edge of tissue such as an inner wall of a left ventricle within a patient's heart or the inner wall of a vessel. Many tissue structures also vary in intensity and thus varying degrees or strengths of edges may be detected within a tissue structure. Examples of edge detection algorithms include, but are not limited to, the Sobel operator and the Difference of Gaussian operator.

At 208 the processor module 116 computes an edge weight value for each of the pixels. The edge weight values are used to modulate the original pixel value, which may also be referred to as the pixel's input intensity value. For example, when an edge is detected the processor module 116 may assign the pixel an edge weight value of 1.0 and when no edge is detected the pixel may be assigned an edge weight value of 0.0. In other words, the intensity values of edge pixels (e.g. pixels associated with an edge) may be unchanged, slightly increased or slightly decreased, while intensity values of non-edge pixels (e.g. pixels not associated with an edge) may be decreased or set to 0.0. In another embodiment, when an edge is detected the processor module 116 may assign the pixel an edge weight value within a range, such as between 0.0 and 1.0, based on a relative strength of the detected edge. For example, an edge that is between tissue and fluid may have a relatively high strength while an edge that is within tissue may have a relatively low strength. In yet another embodiment, edge weight values greater than 1.0 may be used to further emphasize or enhance the detected edges. For example, a maximum edge weight value of 1.5 or greater may be used for the strongest detected edge. In this case, the intensity values of the pixels associated with an edge may be increased in comparison with the input intensity values of the original image data of 200.

At 210 the processor module 116 may compute an intensity weight value that may be based on the input intensity values of the original image data of 200 or the intensity values of the smoothed image data of 204. The intensity weight values may be used together with the edge weight values to modulate the pixel's input intensity value. Each pixel thus has an intensity weight value that is based on the original intensity of the pixel. The input intensity value may be within a range from 0.0 (minimum intensity value), representing a black pixel or no image data, to 1.0 (maximum intensity value), which may represent the maximum intensity. The maximum intensity value may be based on, for example, the range of intensity values within the image data, set by the operator or a protocol, or based on a contrast range of the display 118. Pixels having a low input intensity value may be assigned a low or 0.0 intensity weight value. Pixels having input intensity values that are slightly higher, such as an input intensity of 0.25 or 0.50, may be assigned an intensity weight value of 0.5 and 0.75, respectively. Pixels having intensity values that are relatively high within the range of input intensity values, such as 0.75 and 1.00, may be assigned an intensity weight value of 1.0. The intensity weight values are exemplary only and not limited to the values discussed herein.

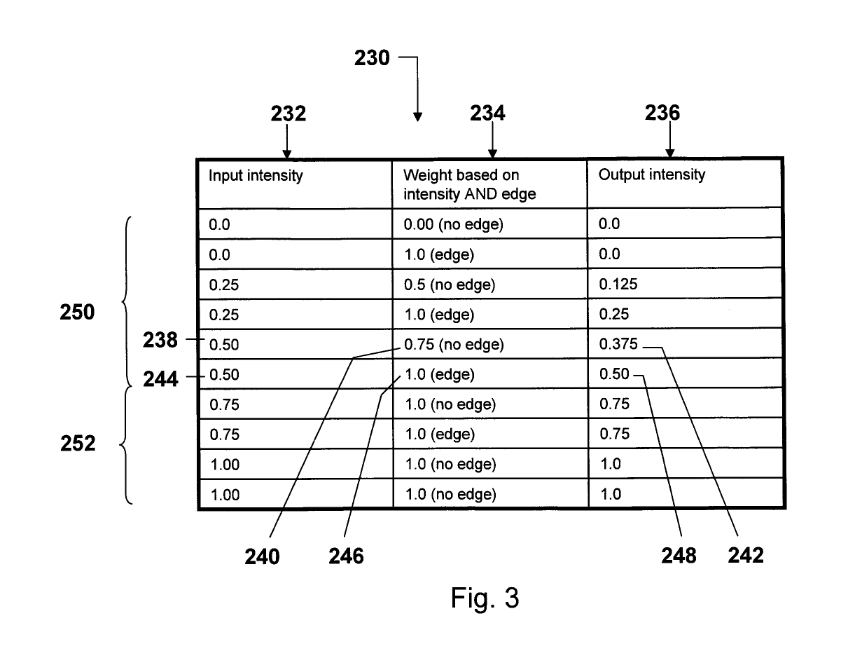

At 212, the processor module 116 may compute a weight value for each pixel or for groups of pixels based on the edge and intensity weight values. FIG. 3 illustrates an exemplary table 230 of several intensity values. Pixel input intensity values 232 represent the pixel values of the original image data of 200 or the smoothed image of 204. It should be understood that many more input intensity values 232 may be used between the range of 0.0 and 1.00. A weight value 234 is based on both the edge weight values of 208 and the intensity weight values of 210. In this example, at each of the input intensity values 232 there is a different weight value 234 based on whether “no edge” or an “edge” is detected for the pixel at 206. As discussed previously, the edge weight values may also be a range of values, and some embodiments may have additional weight values 234 at some or all of the input intensity values 232 based on varying edge strengths. Output intensity values 236 are then computed based on the weight values 234 that take into account both the edge detection as well as the original or smoothed intensity value of the pixel.

For example, a first pixel having an input intensity value 238 (e.g., 0.50) may be assigned a weight value 240 (e.g., 0.75) when no edge is detected at 206. Output intensity value 242 is thus 75 percent of the input intensity value 238, or 0.375. For a second pixel having an input intensity value 244 (e.g., 0.50) that also is an edge, however, a weight value 246 of 1.0 may be assigned. Output intensity value 248 is thus 100 percent of the input intensity value 244, or 0.50. Therefore, when the input intensity value 232 is the same for two different pixels, the input intensity value 238 of a non-edge pixel may be decreased while the input intensity value 244 of an edge pixel remains the same. In another embodiment, the input intensity value 232 of an edge pixel may be increased by having a weight value 234 that is greater than 1.0. Also, for relatively low input intensity values 250, the intensity values of non-edge pixels may be decreased while for relatively high input intensity values 252 the intensity values of non-edge pixels may be unchanged. Although the range of relatively low input intensity values 250 is illustrated at the range of intensity values from 0.0 to 0.50, it should be understood that a different range of values may be used.

Returning to FIG. 2, at 214 the processor module 116 may use the output intensity values 236 of FIG. 3 to form a weighted image. The weighted image has increased intensity or the original intensity of the pixels associated with movement and/or edges. In one embodiment, the weighted image may not be displayed on the display 118. In another embodiment, the processor module 116 may display the weighted image on the display 118 and allow the operator to selectively modify the weighted image. For example, in an alternative embodiment the operator may select a point or area within the image data that is tissue. Tissue structures within the heart may be, for example, the septum or wall between the left and right ventricles and/or valves within the heart. The processor module 116 may then search proximate to the point or area selected by the operator to identify an area of tissue as well as associated boundaries. It should be understood that known edge detection and tissue selection algorithms may be used.

At 216 the processor module 116 temporally filters the weighted image to identify moving structures within the image data. By way of example only, the processor module 116 may compare a first image frame to a second, third or subsequent image frame to identify one or more moving structures or pixels. Also, motion detection may be based on speckle tracking, tissue Doppler imaging, and/or other motion detection algorithms. Pixels or regions of pixels where motion is detected may be further enhanced, such as by increasing the weight value 234 of FIG. 3 associated with the pixel or by selecting a weight value 234 such that the pixel's input intensity value remains unchanged. Therefore, the reduction of intensity may be limited to non-moving pixels. Temporal weighting provides enhancement to tissue areas such as valves. Also, locations of edges may vary over time with movement of the heart and structures within the heart, and thus adjusting the edges temporally provides a more robust detection.

For example, referring again to FIG. 3, a pixel having an input intensity value 232 (e.g., 0.50), no edge, but with motion detected may be assigned a weight value 234 that is greater than 0.75. A pixel having an input intensity value 232 (e.g., 0.50), an edge and motion detected may be assigned a weight value 234 (e.g., 1.0 or greater), depending upon the range of weight values being used. By way of example only, the temporally filtered weighted image may be a pixel representation or mapping wherein regions that are either moving or have edges are bright or have respectively higher intensity values, and regions that are not moving and don't have edges are darker or have respectively lower intensity values. It should be understood that the temporal filtering of 210 may not be applicable to all types of image data, such as some types of vascular imaging.

At 218 the processor module 116 modulates (e.g. adjusts and/or varies) the intensity values of at least a portion of the pixels in the original image data of 200 based on the weight values 234 of the corresponding pixels within the weighted image, which may be temporally filtered, to form a selectively enhanced image. Therefore, intensity values within the original image data may be adjusted or varied based on the edge detection, input intensity value and/or motion detection. In one embodiment, the pixels associated with tissue may be unaltered, while the pixels associated with fluid (or the areas not identified as an edge, tissue or moving tissue) may be reduced in value. In another embodiment, the intensity of the pixels in the original image may be used to decrease the filtering effect in bright regions that are normally tissue regions, maintaining the tissue information. In yet another embodiment, the pixels may be further adjusted based on an additional curve or mapping function that may be selected or modified by the operator.

At 220 the processor module 116 displays the selectively enhanced image(s) on the display 118. The processor module 116 may display the selectively enhanced images in real-time as the ultrasound image data is acquired, or may display the selectively enhanced images based on previously recorded image data, such as in a cine loop. Optionally, the operator may select a portion of the selectively enhanced image with the user interface 124 and manually modulate pixel values and/or apply a greater or lesser weight value to all or a portion of the pixels.

The selective enhancement of FIG. 2 decreases the noise within the areas of the image that represent fluid, such as within the ventricle, while intensifying or leaving unchanged the areas of tissue and areas of movement. In other words, the selectively enhanced image may be darker (have less intensity) than the original image in regions that are not moving and do not contain visible edges and/or tissue. The selectively enhanced image may be the same or brighter (have greater intensity) than the original image in areas that have movement and/or have visible edges and/or tissue.

In another embodiment, blood flow within the image data may be detected. For example, color flow may be used to compute the blood flow in the ventricle. The blood flow data may be used to identify pixels associated with fluid and may be used to adjust the input intensity values 232 and/or weight values 234 of FIG. 3.

In another embodiment, the smoothed and/or weighted images may be displayed on the display 118, allowing the operator to accept, reject or modify the changes. In yet another embodiment, if the image data is acquired over time, the processor module 116 may display one or more of the smoothed and/or weighted images from a predetermined or selected point within the time period over which the image data is acquired for review by the operator.

By reducing the intensity of the noise in fluid, the contrast is increased or enhanced between the fluid and the tissue. This reduction of the intensity of the noise in fluid improves image quality as well as the ability of the operator to perceive the boundaries or edges between the fluid and the tissue. For example, with selective enhancement the robustness of rendering algorithms used with 3D or 4D imaging is increased as noise in the fluid often obstructs the tissue boundary that the operator is trying to see.

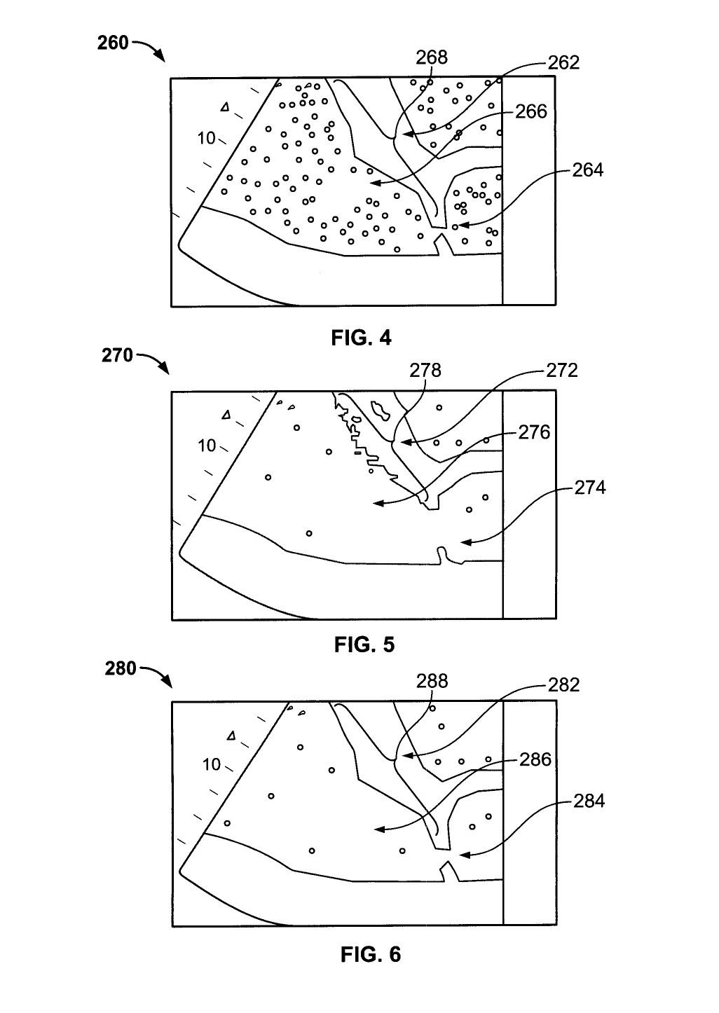

FIG. 4 is a drawing 260 that represents an example of cardiac scanning. The drawing 260 is representative of the original image data of 200 of FIG. 2 and has a tissue area 262 that may represent the septum between the left and right ventricles of a patient's heart. Valve area 264 represents the aortic valve and fluid area 266 may represent the blood within the left ventricle. Edge region 268 of the tissue area 262 illustrates the edge of the tissue next to the fluid area 266. At least a portion of the pixels in the edge region 268 and the valve area 264 are edge pixels. The fluid area 266 has noise there-within that may obscure the wall of the ventricle. The fluid area 266 has non-edge pixels that typically have lower pixel intensities than the pixels representing tissue.

FIG. 5 is a drawing 270 that illustrates an exemplary result of intensity weighting only of the image data of FIG. 4. In this example, the entire image, including tissue area 272, valve area 274 and fluid area 276, is decreased in intensity. Therefore, although noise is reduced throughout the image and within the fluid area 276, data relating to the structures within the tissue area 272 and valve area 274 may be decreased or lost. For example, edge region 278 of the tissue area 272 is now missing image data when compared to the edge region 268 of FIG. 4.

FIG. 6 is a drawing 280 that illustrates intensity weighting, edge detection weighting and temporal filtering of the image data of FIG. 4 according to various embodiments of the invention. The noise within fluid area 286 is decreased while tissue area 282 and valve area 284 have retained or enhanced (increased) the intensity values of the pixels, improving the image quality. Also, edge region 288 has retained the image data of the edge region 268 of FIG. 4.

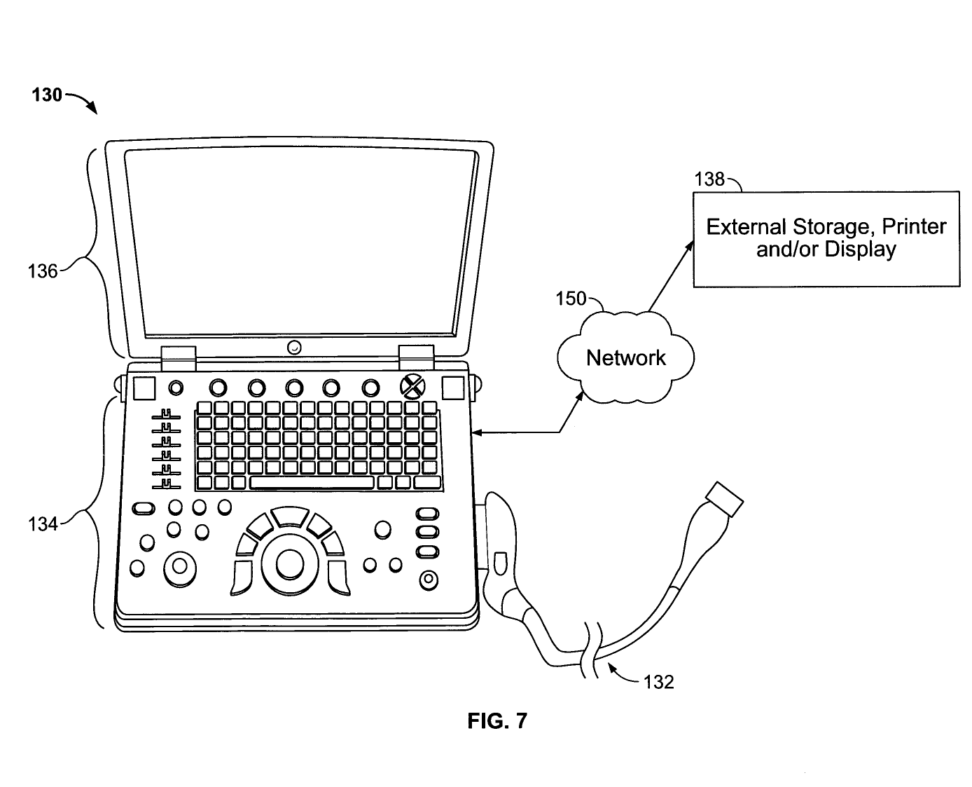

Selective enhancement may be used to enhance the images acquired and/or accessed by any type of ultrasound system. FIG. 7 illustrates a 3D-capable miniaturized ultrasound system 130 having a transducer 132 that may be configured to acquire 3D ultrasonic data. For example, the transducer 132 may have a 2D array of transducer elements 104 as discussed previously with respect to the transducer 106 of FIG. 1. A user interface 134 (that may also include an integrated display 136) is provided to receive commands from an operator. As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. The integrated display 136 (e.g., an internal display) is also provided and is configured to display a medical image.

The ultrasonic data may be sent to an external device 138 via a wired or wireless network 150 (or direct connection, for example, via a serial or parallel cable or USB port). In some embodiments, external device 138 may be a computer or a workstation having a display. Alternatively, external device 138 may be a separate external display or a printer capable of receiving image data from the ultrasound system 130 and of displaying or printing images that may have greater resolution than the integrated display 136.

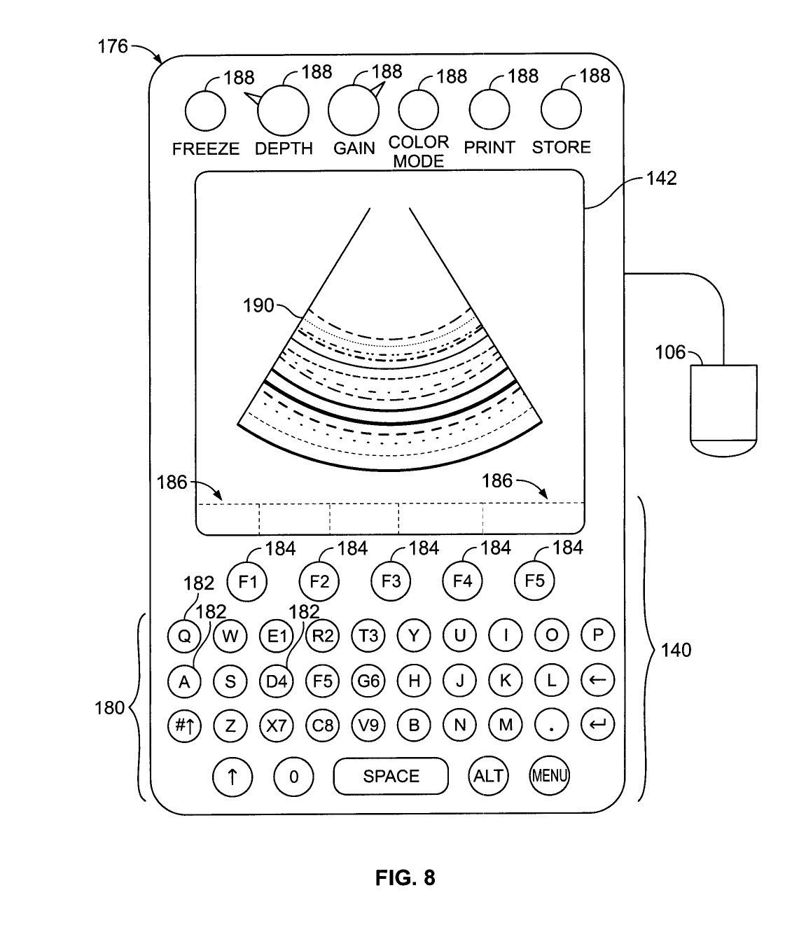

FIG. 8 illustrates a hand carried or pocket-sized ultrasound imaging system 176 wherein display 142 and user interface 140 form a single unit. By way of example, the pocket-sized ultrasound imaging system 176 may be a pocket-sized or hand-sized ultrasound system approximately 2 inches wide, approximately 4 inches in length, and approximately 0.5 inches in depth and weighs less than 3 ounces. The display 142 may be, for example, a 320×320 pixel color LCD display (on which a medical image 190 may be displayed). A typewriter-like keyboard 180 of buttons 182 may optionally be included in the user interface 140. It should be noted that the various embodiments may be implemented in connection with a miniaturized ultrasound system having different dimensions, weights, and power consumption.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 142. The system 176 may also have additional keys and/or controls 188 for special purpose functions, which may include, but are not limited to “freeze,” “depth control,” “gain control,” “color-mode,” “print,” and “store.”

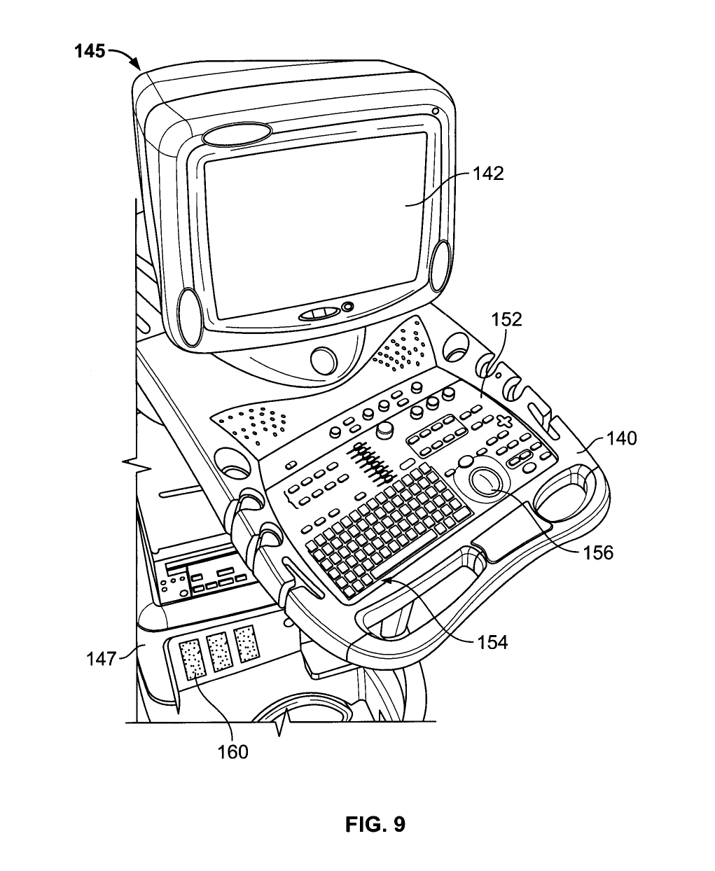

FIG. 9 illustrates a console-based ultrasound imaging system 145 provided on a movable base 147. The ultrasound imaging system 145 may also be referred to as a cart-based system. A display 142 and user interface 140 are provided and it should be understood that the display 142 may be separate or separable from the user interface 140. The user interface 140 may optionally be a touchscreen, allowing the operator to select options by touching displayed graphics, icons, and the like.

The user interface 140 also includes control buttons 152 that may be used to control the ultrasound imaging system 145 as desired or needed, and/or as typically provided. The user interface 140 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided. The system 145 has at least one probe port 160 for accepting probes.

A technical effect of at least one embodiment is the ability to reduce or remove noise from within fluid areas of an image while retaining image data of tissue. Edge detection is used to detect tissue edges within the image data. Edge weight values are determined for each pixel based on whether the pixel is associated with an edge. A higher weight value is assigned for an edge pixel and a range of edge weight values may be provided based on the strength of a particular edge. The weight value for a particular pixel may be further adjusted based on the input intensity value for the pixel. The input intensity value may be from an originally acquired or stored image or a smoothed image. Tissue motion may also be detected within the image data, allowing moving tissue to be identified and enhanced. Therefore, the original intensity values may be modified based on edge and motion detection as well as original intensity values to form a selectively enhanced image that has reduced noise in fluid areas, but that still retains the desired tissue and edge data.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for reducing noise in ultrasound images, comprising:
accessing ultrasound image data comprising at least a fluid area and a tissue area, the image data comprising pixels having input intensity values;
detecting edge pixels associated with an edge within the tissue area; and
modulating the input intensity values of at least a portion of non-edge pixels to be less than the input intensity value of the non-edge pixel to form a selectively enhanced image for display.

2. The method of claim 1, further comprising modulating the input intensity values of the edge pixels to be one of the same as and greater than the input intensity value of the edge pixel.

3. The method of claim 1, wherein the image data is acquired over time, the method further comprising:
detecting movement within the image data; and
modulating the intensity values of the pixels associated with the movement to be one of the same as and greater than the input intensity value of the pixel.

4. The method of claim 1, wherein the image data is acquired over time, the method further comprising:
detecting movement within the image data with at least one of tissue Doppler imaging and speckle tracking; and
modulating the intensity values of the pixels associated with the movement to be one of the same as and greater than the input intensity value of the pixel.

5. The method of claim 1, the modulating further comprising decreasing the input intensity values of the non-edge pixels that have relatively low input intensity values.

6. The method of claim 1, further comprising smoothing the image data to form smoothed image data, wherein the smoothing is based on at least one of speckle reduction and averaging.

7. The method of claim 1, wherein the image data represents at least one of two-dimensional (2D) image data, three-dimensional (3D) image data, 2D image data over time, and 3D image data over time.

8. The method of claim 1, further comprising modulating the input intensity values of the edge pixels to be greater than the input intensity value of the edge pixel when the edge pixel has a relatively high input intensity value.

9. The method of claim 1, further comprising:
detecting blood flow within the image data; and
modulating the input intensity values of at least a portion of the pixels associated with the blood flow to decrease the intensity values of the pixels.

10. A computer readable medium for selectively enhancing image data, comprising:
instructions to access image data comprising at least a fluid area and a tissue area;
instructions to detect at least one edge comprising edge pixels within the tissue area; and
instructions to modulate intensity values associated with at least a portion of non-edge pixels to increase a contrast level between the fluid area and the tissue area.

11. The computer readable medium of claim 10, wherein the image data further comprises a tissue area that moves over time, the computer readable medium further comprising:
instructions to detect movement of pixels within the image data over time; and
instructions to modulate the intensity values of the pixels associated with the movement.

12. The computer readable medium of claim 10, further comprising:
instructions to detect a relative strength of the detected edges; and
instructions to modulate the intensity values of the edge pixels based on the relative strength of the detected edges.

13. The computer readable medium of claim 10, further comprising an ultrasound system configured to acquire the image data, the ultrasound system being one of a miniaturized ultrasound system, a pocket-sized ultrasound imaging system and a console-based ultrasound imaging system.

14. The computer readable medium of claim 10, further comprising instructions to smooth the image data.

15. The computer readable medium of claim 10, wherein the intensity values further comprise a range of intensity values, the computer readable medium further comprising instructions to decrease relatively low intensity values of non-edge pixels.

16. A method for processing image data, comprising:
accessing image data comprising pixels having input intensity values, the input intensity values having a range based on minimum and maximum intensity values;
detecting edge pixels associated with an edge within the image data;
computing a weight value for each of the pixels based on the associated input intensity value;
decreasing the weight values of at least a portion of non-edge pixels; and
displaying an image wherein the pixels have output intensity values based on the weight values and the input intensity values.

17. The method of claim 16, further comprising modulating the weight value of at least one of the edge pixels to obtain an output intensity value that is one of greater than the input intensity value of the edge pixel, equal to the maximum intensity value and greater than the maximum intensity value.

18. The method of claim 16, wherein the image data is acquired over time, the method further comprising:
detecting tissue movement within the image data over time; and
modulating the weight values of the pixels associated with the tissue movement to obtain output intensity values that are one of equal to the input intensity value of the pixel, greater than the input intensity value of the pixel, equal to the maximum intensity value and greater than the maximum intensity value.

19. The method of claim 16, wherein the image data is acquired over time, the method further comprising:
detecting fluid movement within the image data over time; and
modulating the weight values of the pixels associated with the fluid movement to obtain output intensity values that are one of less than the input intensity value of the pixel and equal to the minimum intensity value.

20. The method of claim 16, further comprising acquiring the image data and displaying the image in real-time with respect to the acquisition.
